(19) United States
(12) Patent Application Publication
(10) Pub. No.: US 2026/0369405 A1
(43) Pub. Date: Nov. 19, 2026

(54) SYSTEM AND METHOD FOR PREDICTING LUNG CANCER RISK BASED ON LOW-DOSE CT SCANS

(71) Applicant: UNIVERSITY OF PITTSBURGH-OF THE COMMONWEALTH SYSTEM OF HIGHER EDUCATION, PITTSBURGH, PA (US)

(72) Inventor: JIANTAO PU, SEWICKLEY, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 19/157,050

(22) PCT Filed: Mar. 6, 2024
(86) PCT No.: PCT/US2024/018596

§ 371 Date: Aug. 15, 2025

Related U.S. Application Data
(60) Provisional application No. 63/489,053, filed on Mar. 8, 2023.

(51) Int. Cl. G16H 50/20; G06T 7/00; G06T 7/11; G16H 50/30

(52) U.S. Cl. CPC G16H 50/20; G06T 7/0012; G06T 7/11; G16H 50/30; G06T 2207/10081; G06T 2207/20081; G06T 2207/20084; G06T 2207/30061

(57) ABSTRACT

A system for predicting lung cancer risk for an individual includes a segmentation component configured to receive image data representing a number of LDCT images captured from the individual and process the image data to automatically detect and segment a plurality of tissues and/or tissue structures in the image data, a feature extraction and quantification component configured to extract and quantify a number of features from the detected and segmented tissues and/or tissue structures, wherein the features include one or more of a number of circulatory system features, a number of body composition features, or a number of lung characteristic features, and a model component that implements a trained time-to-event machine learning model, wherein the trained time-to-event machine learning model is configured to receive the number of features and determine a lung cancer risk for the individual based on at least the number of features.

### CROSS REFERENCE TO RELATED APPLICATIONS

This application claims priority to U.S. Provisional Patent Application Ser. No. 63/489,053, filed on Mar. 8, 2023 and titled “System and Method for Predicting Lung Cancer Risk Based on Low-Dose CT Scans”, the disclosure of which is incorporated herein by reference.

### STATEMENT OF GOVERNMENT INTEREST

This invention was made with government support under grant #s CA237277; CA271888, AT012282, and CA047904 awarded by the National Institutes of Health (NIH). The government has certain rights in the invention.

### FIELD OF THE INVENTION

The disclosed concept relates generally to the treatment of lung cancer, and, in particular, to a system and method for predicting the risk of lung cancer in an individual based on low-dose computed tomography (LDCT) images captured from the individual.

### BACKGROUND OF THE INVENTION

Lung cancer is responsible for a staggering 25% of all cancer-related deaths in the United States. The high mortality is partly attributed to the lack of early-stage symptoms and limited access to screening. The National Lung Screening Trial (NLST) showed that low-dose computed tomography (LDCT) scans can reduce lung cancer-related deaths by approximately 20% compared to chest X-rays. As a result, the Center for Medicare and Medicaid Services (CMS) has decided to cover annual lung cancer screening with LDCT scans for asymptomatic adults with a history of tobacco smoking. The NLST findings served as the primary motivation for this decision. In 2021, the U.S. Preventive Services Task Force (USPSTF) updated its recommendation for annual lung screening with LDCT. The updated guidelines recommend annual screening for adults aged 50-80 years with a tobacco smoking history of 20 pack-years or more who either smoked or quit within the past 15 years. The updated recommendation expands the population eligible for LDCT lung screening by approximately 87%, resulting in 14.5 million people in the U.S. being eligible for screening.

Despite the promise of LDCT for lung cancer screening, the results from NLST show that only a tiny percentage of patients who underwent the procedure were diagnosed with lung cancer. Out of the 53,454 smokers enrolled in the study, only 3.85% (2,058 subjects) were diagnosed with lung cancer, indicating that the majority of screened subjects did not develop the disease but are still recommended to have annual LDCT scans. This will not only cause unnecessary radiation exposure to those healthy subjects, but will also impose a significant economic burden on public health and low-income families. Additionally, it is worth mentioning that there are still 16% of women and 10% of men who never smoke but are diagnosed with lung cancer, underscoring the need for identifying additional biomarkers that can accurately assess the risk of developing the disease. The high cost and exposure to unnecessary radiation associated with LDCT scans also highlight the importance of finding more reliable methods for predicting lung cancer risk, by which a large portion (e.g., 30%-50%) of subjects eligible for LDCT lung cancer screening can be excluded from unnecessary annual screening (e.g., subjects with low risk of lung cancer will have an LDCT scan every 2-3 years instead of every year).

### SUMMARY OF THE INVENTION

In one embodiment, the disclosed concept provides a system for predicting lung cancer risk for an individual is provided. The system includes a processing apparatus that includes a segmentation component, a feature extraction and quantification component, and a model component. The segmentation component is structured and configured to: (i) receive image data representing a number of low dose computed tomography (LDCT) images captured from the individual, and (ii) process the image data to automatically detect and segment a plurality of tissues and/or tissue structures in the image data. The feature extraction and quantification component is structured and configured to extract and quantify a number of features from the detected and segmented tissues and/or tissue structures, wherein the features include one or more of a number of circulatory system features, a number of body composition features, or a number of lung characteristic features. The model component implements a trained time-to-event machine learning model, wherein the trained time-to-event machine learning model is structured and configured to receive the number of features and determine a lung cancer risk for the individual based on at least the number of features.

In another embodiment, the disclosed concept provides a method for predicting lung cancer risk for an individual. The method includes receiving image data representing a number of low dose computed tomography (LDCT) images captured from the individual, processing the image data in a processing apparatus to automatically detect and segment a plurality of tissues and/or tissue structures in the image data and extract and quantify a number of features from the detected and segmented tissues and/or tissue structures, wherein the features includes one or more of a number of circulatory system features, a number of body composition features, or a number of lung characteristic features, receiving the number of features in a model component of the processing apparatus, the model component implementing a trained time-to-event machine learning model, and determining in the model component a lung cancer risk for the individual based on at least the number of features.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of an exemplary lung cancer risk prediction system for predicting the risk of lung cancer in an individual based on LDCT images captured from the individual according to an exemplary embodiment of the disclosed concept; and

FIG. 2 is a flowchart showing a method of predicting lung cancer risk based on LDCT images according to an exemplary embodiment of the disclosed concept.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the disclosed concept. It will be evident, however, that the disclosed concept can be practiced without these specific details without departing from the spirit and scope of this innovation.

As described herein, the disclosed concept provides a system and method for personalized LDCT-based lung cancer screening. Given widespread adoption of CT-based lung cancer screening in high-risk individuals, the disclosed concept provides a novel computer tool to quantitatively assess an individual's risk of developing lung cancer over time based on LDCT scans and, in some embodiments, particular patient demographics (e.g., age, gender, race, smoking history). The goal of the disclosed concept is to enable an efficacious personalized LDCT-based lung cancer screening. Competing risk time-to-event statistical and machine learning methods are used to develop models for predicting lung cancer risk function over time, namely the risk of developing lung cancer at a specific time point in the future. In the exemplary embodiment, the predicted risk function over time will be used to stratify a subject into low, moderate, and high risk categories, which may then determine the frequency of lung cancer screening for the individual.

In various exemplary embodiments described herein, one or more of the following three categories of image features will be automatically quantified from chest LDCT scans: (1) circulatory system characteristics, such as artery and vein morphology, coronary artery calcification, aorta calcification, and/or heart volume/morphology, (2) lung characteristics, such as lung density, extent of parenchyma damage (e.g., emphysema, or interstitial lung disease (ILD)), the presence of lung nodules, and/or airway and vessel morphology, and (3) body composition, including one or more of five different body tissues (i.e., subcutaneous adipose tissue (SAT), visceral adipose tissue (VAT), intramuscular adipose tissue (IMAT), skeletal muscle (SM), and bone). The underlying rationale of the disclosed concept is that (1) the lung cancer development cannot be independent of its global and regional environments, and (2) lung cancer is significantly associated with personal life habit (e.g., cigarette smoking), genetic markers, and/or environmental factors (e.g., airborne dust in the workplace), while the long-term impact of these risk factors on health can be faithfully reflected by the changes of body composition and lung characteristics depicted on high resolution CT images. For example, smoking often causes the damage of parenchyma, airway remodeling, and vascular calcification. A close relationship between airway obstruction and lung cancer has been verified, with a conclusion that even a modest reduction in airflow is an independent risk factor for lung cancer.

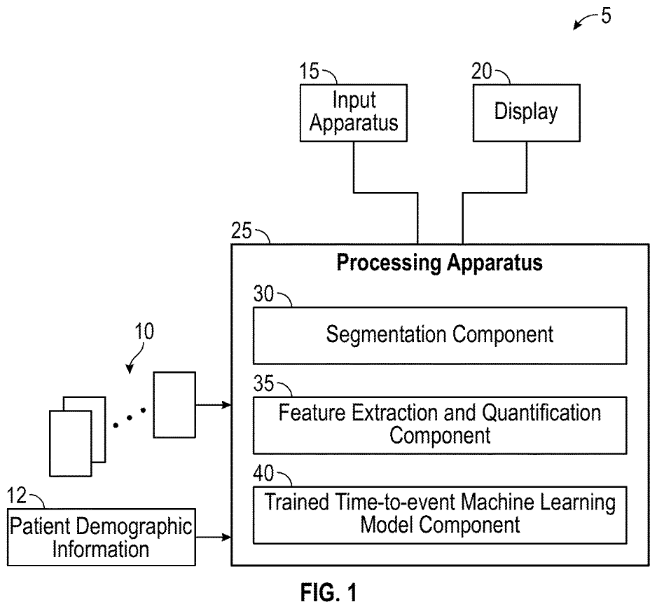

FIG. 1 is a schematic diagram of an exemplary lung cancer risk prediction system 5 structured and configured for predicting the risk of lung cancer in an individual based on LDCT images captured from the individual according to an exemplary embodiment of the disclosed concept as described herein. As seen in FIG. 1, system 5 is a computing device structured and configured to generate and/or receive LCDT image data 10 and patient demographic information 12 in FIG. 1 for an individual and process that data as described herein to determine a predicted lung cancer risk for the individual. System 5 may comprise, for example and without limitation, a PC, a laptop computer, a tablet computer, or any other suitable computing device structured and configured to perform the functionality described herein. System 5 includes an input apparatus 15 (such as a keyboard), a display 20 (such as an LCD), and a processing apparatus 25. A user, such as a caregiver, is able to provide input into processing apparatus 25 using input apparatus 15, and processing apparatus 25 provides output signals to display 20 to enable display 20 to display information to the user relating to the predicted lung cancer risk for the individual as described in detail herein. Processing apparatus 25 comprises a processor and a memory. The processor may be, for example and without limitation, a microprocessor (μP), a microcontroller, an application specific integrated circuit (ASIC), or some other suitable processing device, that interfaces with the memory. The memory can be any one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. The memory has stored therein a number of routines that are executable by the processor, including routines for implementing one or more of the exemplary embodiments of the disclosed concept as described herein.

In particular, processing apparatus 25 includes a segmentation component 30 configured for identifying and segmenting lung and/or heart tissues and/or lung/heart tissue structures in the received LDCT images 10. Segmentation component 30 may employ any known or hereafter developed suitable segmentation approach to automatically segment the tissues and/or tissue structures. Processing apparatus 25 further includes a feature extraction and quantification component 35 configured for extracting and quantifying a plurality of features from the image data and/or the detected and segmented tissues and tissue structures. In one exemplary embodiment, the extracted and quantified features may include a number of circulatory system features (e.g., pulmonary circulatory system (PCS), a number of lung characteristic features, and/or a number of body composition features. Several particular exemplary features in each of these feature categories are described in detail herein.

Processing apparatus 25 also includes a trained time-to-event machine learning model component 40. Trained time-to-event machine learning model component 40 is configured to receive the extracted and quantified features and patient demographic information 12 and determine a predicted lung cancer risk for the individual based on the received features and patient demographic information 12. In one particular embodiment, the trained time-to-event machine learning model component 40 is configured to determine a plurality of risk categories for the patient, such as, without limitation, a low risk category, a moderate risk category, and a high risk category. In one particular non-limiting exemplary implementation, trained time-to-event machine learning model component 40 comprises a convolutional neural network (CNN), and may employ deep learning and/or iterative selective learning (ISL).

Trained time-to-event machine learning model component 40 is trained using certain training data (i.e., truth data) in the form of certain lung cancer patient cohort data. That data is segmented and particular relevant features as described herein are extracted from the segmented data. In addition, that data also includes demographic information as described herein for the subject patients and the lung cancer diagnosis of the subject patients. In the exemplary embodiment, the patient cohort includes current and former smokers. Also in the exemplary embodiment, the training data is expert annotated by a lung cancer expert. As will be understood, once appropriately trained according to appropriate machine learning techniques, trained time-to-event machine learning model component 40 is able to receive as inputs the extracted and quantified features obtained from LDCT images 10 and demographic information 12 for an individual as described herein (according to any of one or more of the described alternative embodiments) and, based thereon, automatically determine and output the predicted risk of lung cancer for the individual. In one particular embodiment, trained time-to-event machine learning model component 40 is configured to determine a computed risk curve for the individual based on the extracted and quantified features obtained from LDCT images 10 (and, in certain embodiments, also based on demographic information 12).

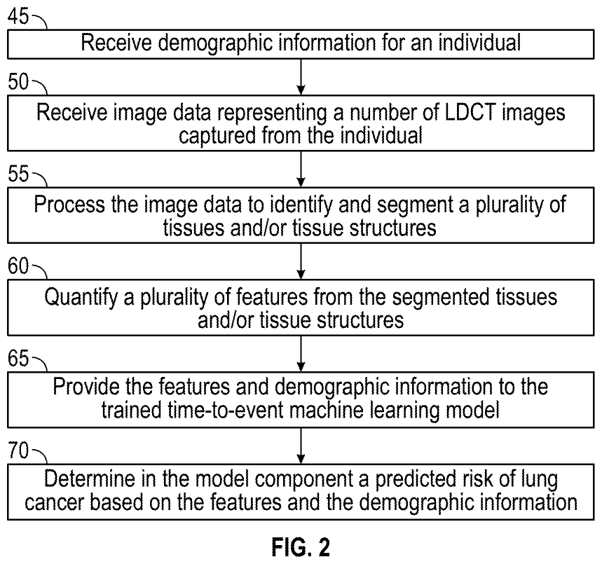

FIG. 2 is a flowchart illustrating a method of predicting the risk of lung cancer in an individual based on LDCT images captured from the individual according to an exemplary embodiment of the disclosed concept. In this exemplary embodiment, the method is implemented in processing apparatus 25 as described, and in particular by segmentation component 30, feature extraction and quantification component 35, and trained time-to-event machine learning model component 40 of processing apparatus 25. It will be understood, however, that this is meant to be exemplary only, and that the method of FIG. 2 may also be implemented in connection with alternative system components.

Referring to FIG. 2, the method begins at step 45, wherein demographic information 12 for an individual is received in processing apparatus 25. Next, at step 50, processing apparatus 25 receives image data representing a number of LDCT images 10 captured from the individual. Then, at step 55, segmentation component 30 processes the image data to identify and segment a plurality of tissues and/or tissue structures in LDCT images 10. Next, at step 60, feature extraction and quantification component 35 extracts and quantifies a plurality of features from the segmented tissues and/or tissue structures. Several particular, nonlimiting exemplary embodiments of the types of features that may be extracted at step 60 and used in the disclosed concept are described herein. The method then proceeds to step 65, wherein the features that have been extracted and quantified and the received patient demographic information 12 are provided to trained time-to-event machine learning model component 40. At step 70, trained time-to-event machine learning model component 40 determines a predicted risk of lung cancer for the individual based on the received features and demographic information. As noted above, in the exemplary embodiment, trained time-to-event machine learning model component 40 determines the risk according to a plurality of risk categories for the individual, such as, without limitation, a low risk category, a moderate risk category, and a high risk category. The predicted risk may be output in a visual format on display 20 for use by a caregiver to treat the individual, such as starting one or more particular treatment regimens for the individual. In addition, in one particular embodiment, processing apparatus 25 is configured to determine a frequency of future lung cancer screenings for the individual based on the determined lung cancer risk for the individual. In one exemplary embodiment, low-risk subjects would be suggested to have a less frequent screening (e.g., after 5 years), high-risk subjects would be suggested to have annual screening, and intermediate risk subjects would be suggested to have a lung screening somewhere in between (e.g., every 2-3 years). The exact frequency of future lung cancer screenings may be determined based on the computed risk curve derived in trained time-to-event machine learning model component 40 from the most recent LDCT scans.

In one particular, non-limiting exemplary embodiment, segmentation component 30 is configured to automatically segment certain PCS structures, including pulmonary arteries and veins, as well as heart regions, in the image data comprising LDCT images 10. Utilizing lung volume segmentation, this embodiment further differentiates intra-and extra-pulmonary arteries and veins. In this particular embodiment, the features that are used to both train and thereafter operate trained time-to-event machine learning model component 40 include: (i) heart volume, (ii) extrapulmonary artery volume, (iii) extrapulmonary vein volume, (iv) intrapulmonary artery volume, and (v) intra-pulmonary vein volume, with the volumes being based on the segmentation results. In addition, in this particular exemplary embodiment, the demographic information includes one or more of: (i) patient age, (ii) patient survival status (censor, cancer, non-cancer death), (iii) emphysema (yes or no), (iv) gender, (v) smoking status (current or former), (vi) race, and (vii) BMI. In one particular implementation, intrapulmonary vein volume is deemed the strongest predictor of future lung cancer and is used as at least one of the features. In another particular implementation, the ratio of intrapulmonary artery volume to the square of the intrapulmonary vein volume is used as one of the features (referred to herein as “intrapulmonary vessel ratio”), as it also shows a strong standalone association with future lung cancer. These particular embodiments may also further include extrapulmonary artery volume as a feature. Furthermore, in one particular exemplary embodiment based on these features, the demographic information that is used in both the training and operation of trained time-to-event machine learning model component 40 includes the presence of emphysema, as it has a strong association with future lung cancer. In addition, age and smoking status may be utilized in conjunction with the presence of emphysema in a particular exemplary embodiment. In one particular exemplary implementation of these embodiments, trained time-to-event machine learning model component 40 uses intrapulmonary vein volume as a feature, and age, smoking status, and the presence of emphysema as demographic information due to the strong prognostic ability of such information.

In other particular exemplary embodiments, the features may include a number of body composition features, a number of lung characteristic features, and/or a number of other circulatory system features. The number of body composition features may include a volume, a density and/or a volume/density ratio with respect to total body tissue volume for a number of body tissues of the individual. The number of body tissues may include or more of subcutaneous adipose tissue (SAT), visceral adipose tissue (VAT), intramuscular adipose tissue (IMAT), skeletal muscle (SM), and bone. The number of lung characteristic features include one or more of lung volume, emphysema volume, airway volume, a ratio of emphysema volume to entire lung volume, and a ratio of airway volume to entire lung volume. The number of circulatory system features may include one or more of heart volume, lung artery volume, lung vein volume as described above and, in addition therefore, one or more of right coronary calcification volume, left coronary calcification volume, aorta calcification volume, and other chest region calcification volumes. In one particular implementation, the demographic information may include the current smoking status for the individual and the clinical emphysema status of the individual, the body composition features may include subcutaneous adipose tissue (SAT) density for the individual, total body mass for the individual, intramuscular adipose tissue (IMAT) ratio for the individual, muscle density for the individual, and/or bone volume for the individual, the lung characteristic features may include lung volume for the individual, emphysema volume for the individual, and FEF25_75 for the individual, and the circulatory system features may include artery volume for the individual and vein volume for the individual. Alternatively, the demographic information may include current smoking status for the individual and clinical emphysema status of the individual, the body composition features may include total body mass for the individual, muscle density for the individual, and bone volume for the individual, the lung characteristic features may include FEF25_75 for the individual, and the circulatory system features may include vein volume for the individual. As a further alternative, the body composition features may further include subcutaneous adipose tissue (SAT) density for the individual and intramuscular adipose tissue (IMAT) ratio for the individual, and the lung characteristic features may further include lung volume for the individual and emphysema ratio for the individual.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A system for predicting lung cancer risk for an individual, comprising:
a processing apparatus, wherein the processing apparatus includes:
a segmentation component structured and configured to: (i) receive image data representing a number of low dose computed tomography (LDCT) images captured from the individual, and (ii) process the image data to automatically detect and segment a plurality of tissues and/or tissue structures in the image data;
a feature extraction and quantification component structured and configured to extract and quantify a number of features from the detected and segmented tissues and/or tissue structures, wherein the features include one or more of a number of circulatory system features, a number of body composition features, or a number of lung characteristic features; and
a model component, the model component implementing a trained time-to-event machine learning model, wherein the trained time-to-event machine learning model is structured and configured to receive the number of features and determine a lung cancer risk for the individual based on at least the number of features.

2. The system according to claim 1, wherein the machine learning model is structured and configured to determine the lung cancer risk for the individual based on the one or more of the number of body composition features, the number of lung characteristic features, the number of circulatory system features and demographic information for the individual.

3. The system according to claim 1, wherein the processing apparatus is structured and configured to determine a frequency of lung cancer screening for the individual based on the lung cancer risk.

4. The system according to claim 3, wherein the processing apparatus is structured and configured to stratify the individual into one of a plurality of risk categories, and wherein the frequency of lung cancer screening is determined based on the one of the plurality of risk categories.

5. The system according to claim 4, wherein the plurality of risk categories include a low risk category, a moderate risk category, and a high risk category.

6. The system according to claim 1, wherein the machine learning model comprises a convolutional neural network (CNN).

7. The system according to claim 6, wherein the CNN employs deep learning.

8. The system according to claim 6, wherein the CNN employs iterative selective learning (ISL).

9. The system according to claim 1, wherein the segmentation component is configured to automatically segment certain pulmonary circulatory system (PCS) structures including one or more of pulmonary arteries, pulmonary veins, or a number of heart regions, and wherein the number of features include one or more of: (i) heart volume, (ii) extrapulmonary artery volume, (iii) extrapulmonary vein volume, (iv) intrapulmonary artery volume, (v) intra-pulmonary vein volume, or (vi) an intrapulmonary vessel ratio in the form of a ratio of the intrapulmonary artery volume to a square of the intrapulmonary vein volume.

10. The system according to claim 9, wherein the number of features includes the intra-pulmonary vein volume.

11. The system according to claim 10, wherein the machine learning model is structured and configured to determine the lung cancer risk based on demographic information for the individual including presence of emphysema.

12. The system according to claim 11, wherein the demographic information includes age and smoking status.

13. The system according to claim 10, wherein the number of features includes the extrapulmonary artery volume.

14. The system according to claim 9, wherein the number of features includes the intrapulmonary vessel ratio.

15. The system according to claim 14, wherein the machine learning model is structured and configured to determine the lung cancer risk based on demographic information for the individual including presence of emphysema.

16. The system according to claim 15, wherein the demographic information includes age and smoking status.

17. The system according to claim 14, wherein the number of features includes the extrapulmonary artery volume.

18. The system according to claim 1, wherein the features include the number of body composition features including a volume, a density and/or a volume/density ratio with respect to total body tissue volume for a number of body tissues of the individual.

19. The system according to claim 18, wherein the number of body tissues include one or more of subcutaneous adipose tissue (SAT), visceral adipose tissue (VAT), intramuscular adipose tissue (IMAT), skeletal muscle (SM), and bone.

20. The system according to claim 1, wherein the features include the number of lung characteristic features including one or more of lung volume, emphysema volume, airway volume, a ratio of emphysema volume to entire lung volume and a ratio of airway volume to entire lung volume.

21. The system according to claim 1, wherein the features include the number of circulatory system features including one or more of heart volume, lung artery volume, lung vein volume, right coronary calcification volume, left coronary calcification volume, aorta calcification volume, and other chest region calcification volume.

22. The system according to claim 2, wherein the demographic information includes current smoking status for the individual and clinical emphysema status of the individual, wherein the body composition features include subcutaneous adipose tissue (SAT) density for the individual, total body mass for the individual, intramuscular adipose tissue (IMAT) ratio for the individual, muscle density for the individual, and bone volume for the individual, wherein the lung characteristic features include lung volume for the individual, emphysema volume for the individual, and FEF25_75 for the individual, and wherein the circulatory system features include artery volume for the individual and vein volume for the individual.

23. The system according to claim 2, wherein the demographic information includes current smoking status for the individual and clinical emphysema status of the individual, wherein the body composition features include total body mass for the individual, muscle density for the individual, and bone volume for the individual, wherein the lung characteristic features include FEF25_75 for the individual, and wherein the circulatory system features include vein volume for the individual.

24. The system according to claim 23, wherein the features include the body composition features further including subcutaneous adipose tissue (SAT) density for the individual and intramuscular adipose tissue (IMAT) ratio for the individual, and wherein the lung characteristic features further include lung volume for the individual and emphysema ratio for the individual.

25. A method for predicting lung cancer risk for an individual, comprising:
receiving image data representing a number of low dose computed tomography (LDCT) images captured from the individual;
processing the image data in a processing apparatus to automatically detect and segment a plurality of tissues and/or tissue structures in the image data and extract and quantify a number of features from the detected and segmented tissues and/or tissue structures, wherein the features includes one or more of a number of circulatory system features, a number of body composition features, or a number of lung characteristic features;
receiving the number of features in a model component of the processing apparatus, the model component implementing a trained time-to-event machine learning model; and
determining in the model component a lung cancer risk for the individual based on at least the number of features.

26. The method according to claim 25, wherein the machine learning model is structured and configured to determine the lung cancer risk for the individual based on the onr or more of the number of body composition features, the number of lung characteristic features, the number of circulatory system features and demographic information for the individual.

27. The method according to claim 25, further comprising determining a frequency of lung cancer screening for the individual based on the lung cancer risk.

28. The method according to claim 27, further comprising stratifying the individual into one of a plurality of risk categories, wherein the frequency of lung cancer screening is determined based on the one of the plurality of risk categories.

29. The method according to claim 28, wherein the plurality of risk categories include a low risk category, a moderate risk category, and a high risk category.

30. The method according to claim 25, wherein the machine learning model comprises a convolutional neural network (CNN) employing iterative selective learning (ISL).

31. The method according to claim 25, wherein the segmentation component is configured to automatically segment certain pulmonary circulatory system (PCS) structures including one or more of pulmonary arteries, pulmonary veins, or a number of heart regions, and wherein the number of features include one or more of: (i) heart volume, (ii) extrapulmonary artery volume, (iii) extrapulmonary vein volume, (iv) intrapulmonary artery volume, (v) intra-pulmonary vein volume, or (vi) an intrapulmonary vessel ratio in the form of a ratio of the intrapulmonary artery volume to a square of the intrapulmonary vein volume.

32. The method according to claim 31, wherein the number of features includes the intra-pulmonary vein volume.

33. The method according to claim 32, wherein the machine learning model is structured and configured to determine the lung cancer risk based on demographic information for the individual including presence of emphysema.

34. The method according to claim 33, wherein the demographic information includes age and smoking status.

35. The method according to claim 32, wherein the number of features includes the extrapulmonary artery volume.

36. The method according to claim 31, wherein the number of features includes the intrapulmonary vessel ratio.

37. The method according to claim 36, wherein the machine learning model is structured and configured to determine the lung cancer risk based on demographic information for the individual including presence of emphysema.

38. The method according to claim 37, wherein the demographic information includes age and smoking status.

39. The method according to claim 36, wherein the number of features includes the extrapulmonary artery volume.

40. The method according to claim 25, wherein the features include the number of body composition features including a volume, a density and/or a volume/density ratio with respect to total body tissue volume for a number of body tissues of the individual.

41. The method according to claim 40, wherein the features include the number of body tissues including or more of subcutaneous adipose tissue (SAT), visceral adipose tissue (VAT), intramuscular adipose tissue (IMAT), skeletal muscle (SM), and bone.

42. The method according to claim 25, wherein the features include the number of lung characteristic features including one or more of lung volume, emphysema volume, airway volume, a ratio of emphysema volume to entire lung volume and a ratio of airway volume to entire lung volume.

43. The method according to claim 25, wherein the features include the number of circulatory system features including one or more of heart volume, lung artery volume, lung vein volume, right coronary calcification volume, left coronary calcification volume, aorta calcification volume, and other chest region calcification volume.

44. The method according to claim 26, wherein the demographic information includes current smoking status for the individual and clinical emphysema status of the individual, wherein the body composition features include subcutaneous adipose tissue (SAT) density for the individual, total body mass for the individual, intramuscular adipose tissue (IMAT) ratio for the individual, muscle density for the individual, and bone volume for the individual, wherein the lung characteristic features include lung volume for the individual, emphysema volume for the individual, and FEF25_75 for the individual, and wherein the circulatory system features include artery volume for the individual and vein volume for the individual.

45. The method according to claim 26, wherein the demographic information includes current smoking status for the individual and clinical emphysema status of the individual, wherein the body composition features include total body mass for the individual, muscle density for the individual, and bone volume for the individual, wherein the lung characteristic features include FEF25_75 for the individual, and wherein the circulatory system features include vein volume for the individual.

46. The method according to claim 45, wherein the body composition features further include subcutaneous adipose tissue (SAT) density for the individual and intramuscular adipose tissue (IMAT) ratio for the individual, and wherein the lung characteristic features further include lung volume for the individual and emphysema ratio for the individual.

47. A computer program product, comprising a non-transitory computer usable medium having a computer readable program code embodied therein, the computer readable program code being adapted to be executed to implement a method of predicting lung cancer risk of an individual over time as recited in claim 25.
